SEQUENCE LISTING (2 sequences)

SEQ ID NO: 1 - protein, 378 aa, Aspergillus oryzae
MGVNFKVLALSALATISHASPLLYPRATDSNVTYVFTNPNGLNFTQMNTTLPNVTIFATGGTIAGSSADNTATTGYKAGAVGIQTLIDAVPEMLNVANVAGVQVTNVGSPDITSDILLRLSKQINEVVCNDPTMAGAVVTHGTDTLEESAFFLDATVNCRKPVVIVGAMRPSTAISADGPLNLLQSVTVAASPKARDRGALIVMNDRIVSAFYASKTNANTVDTFKAIEMGNLGEVVSNKPYFFYPPVKPTGKTEVDIRNITSIPRVDILYSYEDMHNDTLYSAIDNGAKGIVIAGSGSGSVSTPFSAAMEDITTKHNIPIVASTRTGNGEVPSSAESSQIASGYLNPAKSRVLLGLLLAQGKSIEEMRAVFERIGVA

SEQ ID NO: 2 - protein, 378 aa, Aspergillus niger
MPLKPILLSALASLASASPLLYSRTTNETFVFTNANGLNFTQMNTTLPNVTIFATGGTIAGSDSSSTATTGYTSGAVGVLSLIDAVPSMLDVANVAGVQVANVGSEDITSDILISMSKKLNRVVCEDPTMAGAVITHGTDTLEETAFFLDATVNCGKPIVIVGAMRPSTAISADGPFNLLEAVTVAASTSARDRGAMVVMNDRIASAYYVTKTNANTMDTFKAMEMGYLGEMISNTPFFFYPPVKPTGKVAFDITNVTEIPRVDILFSYEDMHNDTLYNAISSGAQGIVIAGAGAGGVTTSFNEAIEDVINRLEIPVVQSMRTVNGEVPLSDVSSDTATHIASGYLNPQKSRILLGLLLSQGKNITEIADVFALGTDA

(19) United States
(12) Patent Application Publication
(10) Pub. No.: US 2015/0320089 A1
(43) Pub. Date: Nov. 12, 2015

(54) Method for Producing a Food Product

(71) Applicant: NOVOZYMES A/S, Bagsvaerd (DK)

(72) Inventor: Hanne Vang Hendriksen, Holte (DK)

(21) Appl. No.: 14/648,968

(22) PCT Filed: Dec. 19, 2013
(86) PCT No.: PCT/EP2013/077417

§ 371 Date: Jun. 2, 2015

Related U.S. Application Data
(60) Provisional application No. 61/746,212, filed on Dec. 27, 2012.

(30) Foreign Application Priority Data
Dec. 21, 2012 (EP) 12198889.3

(51) Int. Cl. A23L 1/015; C12N 9/82; A23F 5/02; A23L 1/216; A23L 1/105

(52) U.S. Cl. CPC A23L 1/0153; A23L 1/216; A23L 1/1055; A23F 5/02; C12N 9/82; A23V 2002/00; C12Y 305/01001

(57) ABSTRACT

The present invention relates to a method for producing a heat-treated food product from a food material which has been contacted with an asparaginase in the presence of magnesium or manganese ions.

### REFERENCE TO SEQUENCE LISTING

This application contains a Sequence Listing in computer readable form. The computer readable form is incorporated herein by reference.

### FIELD OF THE INVENTION

The present invention relates to a method for producing a heat-treated food product from a food material which has been contacted with an asparaginase.

### BACKGROUND OF THE INVENTION

It is well known that acrylamide formation in heated food products may be reduced by a treatment reducing the amount of asparagine in the food materials, such as by subjecting the food materials to the action of the enzyme asparaginase (see e.g. WO2004/026042).

US2007/0141225 relates to reduction of acrylamide in food by exposing the food product to two or more acrylamide-reducing agents. The patent application discloses, for example, use of asparaginase in combination with divalent or trivalent cations. Suggested cations include calcium, magnesium, aluminium, iron, copper and zinc, preferably calcium in the form of calcium chloride.

### SUMMARY OF THE INVENTION

The present inventors have surprisingly found that the activity of asparaginase is increased in the presence of low concentrations of some cations, in particular in the presence of magnesium or manganese ions.

The invention therefore provides a method for producing a heat-treated food product comprising:

(a) contacting of a food material with asparaginase in the presence of magnesium or manganese ions at a concentration of about 0.1-12 millimoles per kg; and
(b) heat-treating the asparaginase treated food material to obtain the heat-treated food product.

### DETAILED DESCRIPTION OF THE INVENTION

SEQ ID NO: 1 is the amino acid sequence of asparaginase from Aspergillus oryzae.

In the context of the present invention, the term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C terminal and/or N terminal amino acid) expressed by the same polynucleotide.

Based on N-terminal sequencing and mass spectrometry (MS) analysis, it seems that N-terminal processing of the asparaginase of SEQ ID NO: 1 is quite heterogeneous. In one embodiment, the mature polypeptide is amino acids 20 to 378 of SEQ ID NO: 1 based on SignalP (Nielsen et al., 1997, Protein Engineering 10: 1-6) that predicts that amino acids 1 to 19 of SEQ ID NO: 1 are a signal peptide.

SEQ ID NO: 2 is the amino acid sequence of asparaginase from Aspergillus niger. In one embodiment, the mature polypeptide of SEQ ID NO: 2 is amino acids 18 to 378 of SEQ ID NO: 2 based on the predicted signal peptide being amino acids 1-17.

The invention provides a method for producing a heat-treated food product comprising:

(a) contacting of a food material with asparaginase in the presence of magnesium or manganese ions at a concentration of about 0.1-12 millimoles per kg; and
(b) heat-treating the asparaginase treated food material to obtain the heat-treated food product.

The method of the invention is relevant for the production of heat-treated food products which are made from a food material which is to be contacted with asparaginase by mixing the asparaginase into the food material. This is the case for, e.g., dough products (bread, crackers, corn chips, etc.), breakfast cereals, mashed potatoes, etc. The method of the invention is also relevant for the production of heat-treated food products which are made from a food material which is to be contacted with asparaginase by the food product being immersed into or sprayed with an asparaginase solution. This is the case for, e.g., food products prepared from cuts of vegetables, such as French fries or sliced potato chips, or for coffee-based food product, e.g., roasted coffee beans or coffee prepared by extraction therefrom, where the green coffee beans are soaked in an asparaginase solution.

It is to be understood that the concentration of magnesium or manganese ions is given in millimoles per kg composition comprising asparaginase. I.e., if the asparaginase and the magnesium or manganese ions are mixed into the food material, the concentration of magnesium or manganese ions is millimoles per kg food material. If the food material is immersed into or sprayed with an asparaginase solution comprising magnesium or manganese ions, the concentration of magnesium or manganese ions is millimoles per kg asparaginase solution. In the latter case, the concentration of magnesium or manganese ions per kg food material is less relevant since only the surface of the food material is contacted with asparaginase.

In one embodiment, the concentration of magnesium or manganese ions is about 0.1-10 millimoles per kg, such as about 0.1-8, 0.1-6, 0.1-4 or 0.1-2 millimoles per kg.

In another embodiment, the concentration of magnesium or manganese ions is about 0.2-12 millimoles per kg, such as about 0.5-12, 1-12, 1.5-12 or 2-12 millimoles per kg.

In another embodiment, the concentration of magnesium or manganese ions is about 0.2-10 millimoles per kg, such as about 0.5-8, 0.5-4, 1-4 or 2-4 millimoles per kg.

In a preferred embodiment, the food material is contacted with asparaginase in the presence of magnesium ions at a concentration of about 0.1-12 millimoles per kg, such as about 0.1-10, 0.1-8, 0.1-6, 0.1-4 or 0.1-2 millimoles per kg. In another preferred embodiment, the food material is contacted with asparaginase in the presence of magnesium ions at a concentration of about 0.2-12 millimoles per kg, such as about 0.5-12, 1-12, 1.5-12 or 2-12 millimoles per kg. In another preferred embodiment, the food material is contacted with asparaginase in the presence of magnesium ions at a concentration of about 0.2-10 millimoles per kg, such as about 0.5-8, 0.5-4, 1-4 or 2-4 millimoles per kg. The magnesium ions may be from any source. They may, e.g., be provided by the addition of MgCl₂.

In another embodiment, the food material is contacted with asparaginase in the presence of manganese ions at a concentration of about 0.1-12 millimoles per kg, such as about 0.1-10, 0.1-8, 0.1-6, 0.1-4 or 0.1-2 millimoles per kg. In another embodiment, the food material is contacted with asparaginase in the presence of manganese ions at a concentration of about 0.2-12 millimoles per kg, such as about 0.5-12, 1-12, 1.5-12 or 2-12 millimoles per kg. In another embodiment, the food material is contacted with asparaginase in the presence of manganese ions at a concentration of about 0.2-10 millimoles per kg, such as about 0.5-8, 0.5-4, 1-4 or 2-4 millimoles per kg. The manganese ions may be from any source. They may, e.g., be provided by the addition of MnO₄S.

The food material which is to be treated according to the method of the invention may be any raw material which is to be included in the food product, or it may be any intermediate form of the food product which occurs during the production process prior to the heating step to obtain the heat-treated food product. It may be any individual raw material used and/or any mixture thereof and/or any mixture thereof also including additives and/or processing aids, and/or any subsequently processed form thereof.

The food product may be made from at least one raw material that is of plant origin, for example a vegetable tuber or root, such as but not limited to the group consisting of potato, carrot, beet, parsnip, parsley root, celery root, sweet potato, yams, yam bean, Jerusalem artichoke, radish, turnip, chicory root and cassava potato; cereal, such as but not limited to the group consisting of wheat, rice, corn, maize, rye, barley, buckwheat, sorghum and oats; coffee; cocoa; chicory; olives; prunes or raisins. Also food products made from more than one raw material are included in the scope of this invention, for example food products comprising both wheat (e.g., in the form of wheat flour) and potato.

Raw materials as cited above are known to contain substantial amounts of asparagine which is involved in the formation of acrylamide during the heating step of the production process. Alternatively, the asparagine may originate from other sources than the raw materials, e.g., from protein hydrolysates, such as yeast extracts, soy hydrolysate, casein hydrolysate or the like, which are used as an additive in the food production process.

The asparaginase is to be added to the food material in an amount that is effective in reducing the level of asparagine present in the food material. This will result in less acrylamide being formed in the heating step which is to take place after the enzyme treatment. Such methods are disclosed, e.g., in WO04/026043. The methods disclosed in WO04/026043 and all preferences disclosed are incorporated by reference.

After the enzyme treatment, the enzyme treated food material is subjected to a heat treatment. The heat treatment is a part of the method for producing a food product from the food material (i.e., the raw material or an intermediate form of the food product). In a conventional method, i.e., a method without asparaginase treatment, more acrylamide would be formed during the heat treatment as compared to the method of the invention where some of the asparagine of the food material is hydrolysed by the asparaginase.

Preferred heating steps are those at which at least a part of an intermediate form of the food product, e.g., the surface of the food product, is exposed to temperatures at which the formation of acrylamide is promoted, e.g. 110° C. or higher, or 120° C. or higher. The heating step in the method according to the invention may be carried out in ovens, for instance at a temperature of 180-250° C., such as for the baking of bread and other bakery products, or in oil such as the frying of potato chips or French fries, for example at 160-195° C. Or it may be carried out by toasting or roasting, such as by toasting of breakfast cereals or by roasting of coffee beans.

In a preferred embodiment, the acrylamide content of the heat-treated food product is reduced by at least 25%, preferably at least 30%, at least 35%, at least 40%, at least 45% or at least 50%, compared to the acrylamide content of a heat-treated vegetable-based food product produced by a similar method without the addition of asparaginase.

In one embodiment of the invention, the heat-treated food product is a cereal-based dough product. It may be a baked cereal-based dough product, such as, e.g., bread, crisp bread, crackers, biscuits, pastry, cake, pretzels, bagels, Dutch honey cake, cookies, gingerbread, ginger cake or baked dough-based chips. Or it may be a fried cereal-based dough product, such as, e.g., corn chips, tortilla chips or taco shells. Cereals may be defined as grasses which are cultivated for the edible components of their grains. In one embodiment, the cereal-based dough product comprises at least one of wheat, rice, corn, maize, rye, barley, buckwheat, sorghum and/or oats. A cereal-based dough may be defined as any mixture comprising at least one cereal-based ingredient and a consumable liquid, with a consistency suitable to be formed into a food product having a definite shape, either by forming the dough directly into such shape or by pouring the dough into a form prior to baking. The food material which is to be contacted with asparaginase may be one or more cereal-based ingredients (for example wheat flour or processed corn), the initial mixture thereof with other ingredients, such as for example water, oil, salt, yeast and/or bread improving compositions, the mixed dough or the corn masa, the kneaded dough, the leavened dough or the partially baked or fried dough or corn masa. The food material may be contacted with asparaginase at a concentration of 50-10,000 ASNU per kg dry matter, more preferably 50-8,000 ASNU per kg dry matter, more preferably 100-7,500 ASNU per kg dry matter and most preferably 100-5,000 ASNU per kg dry matter. The food material is to be contacted with asparaginase in the presence of magnesium or manganese ions, preferably magnesium ions, at a concentration of about 0.1-12 millimoles per kg food material. In a preferred embodiment, the food material is contacted with asparaginase in the presence of magnesium or manganese ions, preferably magnesium ions, at a concentration of about 0.1-10 millimoles per kg food material, such as about 0.1-8, 0.1-6, 0.1-4 or 0.1-2 millimoles per kg food material. In another preferred embodiment, the food material is contacted with asparaginase in the presence of magnesium or manganese ions, preferably magnesium ions, at a concentration of about 0.2-12 millimoles per kg food material, such as about 0.5-12, 1-12, 1.5-12 or 2-12 millimoles per kg food material. In another preferred embodiment, the food material is contacted with asparaginase in the presence of magnesium or manganese ions, preferably magnesium ions, at a concentration of about 0.2-10 millimoles per kg food material, such as about 0.5-8, 0.5-4, 1-4 or 2-4 millimoles per kg food material.

In another embodiment of the invention, the heat-treated food product is a breakfast cereal. The food material which is to be contacted with asparaginase may be, e.g., whole wheat flour, oat flour, corn flour, wheat kernels, oat kernels or oat flakes. The contacting with asparaginase may be performed by mixing the asparaginase into the food material. The food material may be contacted with asparaginase at a concentration of 50-10,000 ASNU per kg dry matter, more preferably 50-8,000 ASNU per kg dry matter, more preferably 100-7,500 ASNU per kg dry matter and most preferably 100-5,000 ASNU per kg dry matter. The food material is to be contacted with asparaginase in the presence of magnesium or manganese ions, preferably magnesium ions, at a concentration of about 0.1-12 millimoles per kg food material. In a preferred embodiment, the food material is contacted with asparaginase in the presence of magnesium or manganese ions, preferably magnesium ions, at a concentration of about 0.1-10 millimoles per kg food material, such as about 0.1-8, 0.1-6, 0.1-4 or 0.1-2 millimoles per kg food material. In another preferred embodiment, the food material is contacted with asparaginase in the presence of magnesium or manganese ions, preferably magnesium ions, at a concentration of about 0.2-12 millimoles per kg food material, such as about 0.5-12, 1-12, 1.5-12 or 2-12 millimoles per kg food material. In another preferred embodiment, the food material is contacted with asparaginase in the presence of magnesium or manganese ions, preferably magnesium ions, at a concentration of about 0.2-10 millimoles per kg food material, such as about 0.5-8, 0.5-4, 1-4 or 2-4 millimoles per kg food material. The heat-treatment of the asparaginase treated food material may be performed by toasting. Toasting may be defined as heating by exposure to radiant heat.

In another embodiment of the invention, the heat-treated food product is a potato-based food product, where the food material to be contacted with asparaginase is mashed potato, a potato-based dough or a suspension of a dehydrated potato product, such as potato flakes or granules. Such food product may be, e.g., dough-based potato snacks, fabricated potato products or croquettes. The food material may be contacted with asparaginase at a concentration of 100-10,000 ASNU per kg dry matter, more preferably 250-8,000 ASNU per kg dry matter, more preferably 500-7,500 ASNU per kg dry matter and most preferably 1,000-7,500 ASNU per kg dry matter. The food material is to be contacted with asparaginase in the presence of magnesium or manganese ions, preferably magnesium ions, at a concentration of about 0.1-12 millimoles per kg food material. In a preferred embodiment, the food material is contacted with asparaginase in the presence of magnesium or manganese ions, preferably magnesium ions, at a concentration of about 0.1-10 millimoles per kg food material, such as about 0.1-8, 0.1-6, 0.1-4 or 0.1-2 millimoles per kg food material. In another preferred embodiment, the food material is contacted with asparaginase in the presence of magnesium or manganese ions, preferably magnesium ions, at a concentration of about 0.2-12 millimoles per kg food material, such as about 0.5-12, 1-12, 1.5-12 or 2-12 millimoles per kg food material. In another preferred embodiment, the food material is contacted with asparaginase in the presence of magnesium or manganese ions, preferably magnesium ions, at a concentration of about 0.2-10 millimoles per kg food material, such as about 0.5-8, 0.5-4, 1-4 or 2-4 millimoles per kg food material. The heat-treatment of the asparaginase treated food material may be performed by frying or baking or a combination thereof.

In another embodiment of the invention, the heat-treated food product is a food product made from cuts of potatoes or other root vegetables such as, but not limited to, carrot, beet, parsnip, parsley root and celery root, which are fried and/or baked. Examples of such food products are French fries, sliced potato chips and sliced chips from root vegetables such as, but not limited to, carrot, beet, parsnip, parsley root and celery root. The food material which is to be contacted with asparaginase may be cuts of potatoes or other root vegetables which have optionally been peeled and/or blanched. Preferably, the cuts of potatoes or other root vegetables have been blanched. The contacting with asparaginase may be performed by the cuts of potatoes or other root vegetables being dipped in, incubated in or sprayed with an asparaginase solution which comprises magnesium or manganese ions, preferably magnesium ions, at a concentration of about 0.1-12 millimoles per kg solution. The asparaginase solution may comprise asparaginase at a concentration of 1,000 to 100,000 ASNU/L, preferably 5,000 to 50,000 ASNU/L, more preferably 10,000 to 30,000 ASNU/L. In a preferred embodiment, the asparaginase solution comprises magnesium or manganese ions, preferably magnesium ions, at a concentration of about 1-12 mM, such as about 1-10, 1-8, 1-6 or 1-4 mM. In another preferred embodiment, the asparaginase solution comprises magnesium or manganese ions, preferably magnesium ions, at a concentration of about 2-12 mM, such as about 2-10, 2-8, 2-6 or 2-4 mM. The heat-treatment of the asparaginase treated food material may be performed by frying or baking or a combination thereof.

In another embodiment of the invention, the heat-treated food product is French fries. The food material which is to be contacted with asparaginase may be cuts of potatoes in the form of wedges or sticks which are of a size and shape suitable for further processing into French fries. In the context of the present invention, French fries is meant to encompass both the final fries ready for consumption and a par-fried pre-product which is to be finally fried or baked before being consumed. Also, French fries is meant to encompass both French fries made from potato sticks and larger French fries made from, e.g., potato wedges. The cuts of potatoes, such as the potato sticks or wedges, may have been blanched before step (a). Blanching may be performed by any method known in the art, e.g., by wet blanching, steam blanching, microwave blanching or infrared blanching. The contacting with asparaginase may be performed by the cuts of potatoes being dipped in, incubated in or sprayed with an asparaginase solution which comprises magnesium or manganese ions, preferably magnesium ions, at a concentration of about 0.1-12 millimoles per kg solution. The asparaginase solution may comprise asparaginase at a concentration of 1,000 to 100,000 ASNU/L, preferably 5,000 to 50,000 ASNU/L, more preferably 10,000 to 30,000 ASNU/L. In a preferred embodiment, the asparaginase solution comprises magnesium or manganese ions, preferably magnesium ions, at a concentration of about 1-12 mM, such as about 1-10, 1-8, 1-6 or 1-4 mM. In another preferred embodiment, the asparaginase solution comprises magnesium or manganese ions, preferably magnesium ions, at a concentration of about 2-12 mM, such as about 2-10, 2-8, 2-6 or 2-4 mM. The cuts of potatoes, such as the potato sticks or wedges, may further be contacted with (such as by dipping in or spraying with) other substances, e.g., sodium acid pyrophosphate (SAPP) and/or glucose, either before, at the same time or after the contacting with asparaginase. The cuts of potatoes, such as the potato sticks or wedges, may optionally be dried. The drying may take place before, at the same time or after the contacting with the asparaginase. In a preferred embodiment, the drying is performed under conditions where the asparaginase is active. I.e., the contacting with asparaginase is to take place before or during the drying. Drying may be performed in a drier with air circulation where temperature, humidity and/or air flow can be adjusted to the level(s) desired. The heat-treatment of the asparaginase treated food material may be performed by frying or baking or a combination thereof.

In another embodiment of the invention, the heat-treated food product is sliced potato chips. The food material which is to be contacted with asparaginase is sliced potatoes having a size which is suitable for further processing into potato chips. Preferably, the sliced potatoes have been blanched. The contacting with asparaginase may be performed by the sliced potatoes being dipped in, incubated in or sprayed with an asparaginase solution which comprises magnesium or manganese ions, preferably magnesium ions, at a concentration of about 0.1-12 millimoles per kg solution. The asparaginase solution may comprise asparaginase at a concentration of 1,000 to 100,000 ASNU/L, preferably 5,000 to 50,000 ASNU/L, more preferably 10,000 to 30,000 ASNU/L. In a preferred embodiment, the asparaginase solution comprises magnesium or manganese ions, preferably magnesium ions, at a concentration of about 1-12 mM, such as about 1-10, 1-8, 1-6 or 1-4 mM. In another preferred embodiment, the asparaginase solution comprises magnesium or manganese ions, preferably magnesium ions, at a concentration of about 2-12 mM, such as about 2-10, 2-8, 2-6 or 2-4 mM. The heat-treatment of the asparaginase treated food material may be performed by frying.

In another embodiment of the invention, the heat-treated food product is a coffee-based food product, e.g., roasted coffee beans or coffee obtained by extraction of the roasted coffee beans. The food material which is to be contacted with asparaginase may be the green coffee beans which have optionally been steamed. The contacting with asparaginase may be performed by soaking of the green coffee beans in a solution comprising asparaginase and magnesium or manganese ions, preferably magnesium ions, at a concentration of about 0.1-12 millimoles per kg solution. The asparaginase solution may comprise asparaginase at a concentration of 500 to 50,000 ASNU/L, preferably 750 to 10,000 ASNU/L, more preferably 1,000 to 7,500 ASNU/L. In a preferred embodiment, the asparaginase solution comprises magnesium or manganese ions, preferably magnesium ions, at a concentration of about 1-12 mM, such as about 1-10, 1-8, 1-6 or 1-4 mM. In another preferred embodiment, the asparaginase solution comprises magnesium or manganese ions, preferably magnesium ions, at a concentration of about 2-12 mM, such as about 2-10, 2-8, 2-6 or 2-4 mM. After the soaking in the asparaginase solution, the coffee beans may be dried. The heat-treatment of the asparaginase treated food material may be performed by roasting to obtain the roasted coffee beans.

Food products obtained by a method of the invention are characterized by significantly reduced acrylamide levels in comparison with equivalent food products obtainable by a production method that does not comprise adding an asparaginase in an amount that is effective in reducing the level of asparagine involved in the formation of acrylamide during a heating step.

In another aspect, the invention provides food products obtainable by a method of the invention as described above.

### Asparaginase

An asparaginase in the context of the present invention means an enzyme having asparaginase activity, i.e., an enzyme that catalyzes the hydrolysis of asparagine to aspartic acid (EC 3.5.1.1).

Asparaginase activity may be determined according to one of the asparaginase activity assays described in the Examples, e.g., by the ASNU assay. In one embodiment, an asparaginase to be used in the method of the present invention has at least 20%, e.g., at least 40%, at least 50%, at least 60%, at least 70%, at least 80%, at least 90%, at least 95%, or at least 100% of the asparaginase activity of the mature polypeptide of SEQ ID NO: 1 when measured at pH 7 and at 37° C. The asparaginase activity may be determined per microgram asparaginase enzyme.

The asparaginase may be obtained from any source, e.g., from a microorganism, from a plant or from an animal.

The asparaginase may be obtained from a microorganism of any genus, e.g., from a bacterium, an archaeon or a fungus. For purposes of the present invention, the term “obtained from” as used herein in connection with a given source shall mean that the asparaginase encoded by a polynucleotide is produced by the source or by a strain in which the polynucleotide from the source has been inserted.

It may be a wild type asparaginase, i.e., an asparaginase found in nature, or it may be a variant asparaginase, i.e., an asparaginase comprising an alteration, i.e., a substitution, insertion, and/or deletion, at one or more (e.g., several) positions compared to a parent asparaginase from which it may have been derived. A substitution means replacement of the amino acid occupying a position with a different amino acid; a deletion means removal of the amino acid occupying a position; and an insertion means adding an amino acid adjacent to and immediately following the amino acid occupying a position.

The asparaginase or its parent, preferably the asparaginase, may be a bacterial asparaginase. For example, the asparaginase may be a Gram-positive bacterial asparaginase such as a Bacillus, Clostridium, Enterococcus, Geobacillus, Lactobacillus, Lactococcus, Oceanobacillus, Staphylococcus, Streptococcus, or Streptomyces asparaginase, or a Gram-negative bacterial asparaginase such as a Campylobacter, E. coli, Flavobacterium, Fusobacterium, Helicobacter, Ilyobacter, Neisseria, Pseudomonas, Salmonella, or Ureaplasma asparaginase.

In one embodiment, the asparaginase is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis asparaginase.

In another embodiment, the asparaginase is a Streptococcus equisimilis, Streptococcus pyogenes, Streptococcus uberis, or Streptococcus equi subsp. Zooepidemicus asparaginase.

In another embodiment, the asparaginase is a Streptomyces achromogenes, Streptomyces avermitilis, Streptomyces coelicolor, Streptomyces griseus, or Streptomyces lividans asparaginase.

The asparaginase or its parent, preferably the asparaginase, may be from an archaeon. For example, the asparaginase may be a Pyrococcus asparaginase, such as, e.g., a Pyrococcus furiosus asparaginase.

In a preferred embodiment, the asparaginase or its parent, preferably the asparaginase, is a fungal asparaginase. For example, the asparaginase may be a yeast asparaginase such as a Candida, Kluyveromyces, Pichia, Saccharomyces, Schizosaccharomyces, or Yarrowia asparaginase; or a filamentous fungal asparaginase such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria asparaginase.

In one embodiment, the asparaginase is a Saccharomyces carlsbergensis, Saccharomyces cerevisiae, Saccharomyces diastaticus, Saccharomyces douglasfi, Saccharomyces kluyveri, Saccharomyces norbensis, or Saccharomyces oviformis asparaginase.

In another embodiment, the asparaginase is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonaturn, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride asparaginase.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The asparaginase may be identified and obtained from other sources including microorganisms isolated from nature (e.g., soil, composts, water, etc.) or DNA samples obtained directly from natural materials (e.g., soil, composts, water, etc.). Techniques for isolating microorganisms and DNA directly from natural habitats are well known in the art. A polynucleotide encoding the asparaginase may then be obtained by similarly screening a genomic DNA or cDNA library of another microorganism or mixed DNA sample. Once a polynucleotide encoding an asparaginase has been detected, the polynucleotide can be isolated or cloned by utilizing techniques that are known to those of ordinary skill in the art (see, e.g., Sambrook et al., 1989, Molecular Cloning, A Laboratory Manual, 2d edition, Cold Spring Harbor, New York).

In one preferred embodiment, the asparaginase or its parent, preferably the asparaginase, is obtained from Aspergillus, e.g., from Aspergillus aculeatus, Aspergillus awamori, Aspergillus foetidus, Aspergillus fumigatus, Aspergillus japonicus, Aspergillus nidulans, Aspergillus niger, Aspergillus oryzae or Aspergillus terreus.

In another preferred embodiment, the asparaginase or its parent, preferably the asparaginase, is obtained from Aspergillus oryzae, e.g., the asparaginase of SEQ ID NO: 1 or the mature polypeptide thereof. In another preferred embodiment, the asparaginase or its parent, preferably the asparaginase, is obtained from Aspergillus niger, e.g., the asparaginase of SEQ ID NO: 2 or the mature polypeptide thereof.

In another preferred embodiment, the asparaginase has at least 50% sequence identity to the mature polypeptide of any one of SEQ ID NOs: 1 or 2, such as at least 60%, at least 70%, at least 80%, at least 90%, at least 95% or at least 98% sequence identity to the mature polypeptide of any one of SEQ ID NOs: 1 or 2.

In another preferred embodiment, the asparaginase is a variant of a parent asparaginase having at least 50% sequence identity to the mature polypeptide of any one of SEQ ID NOs: 1 or 2, such as at least 60%, at least 70%, at least 80%, at least 90%, at least 95% or at least 98% sequence identity to the mature polypeptide of any one of SEQ ID NOs: 1 or 2.

In another preferred embodiment, the asparaginase is an asparaginase disclosed in either of WO2008/110513, WO2008/128974, WO2008/128975, WO2011/134916 or PCT/EP2013/067079, which are hereby incorporated by reference.

In another preferred embodiment, the asparaginase comprises at most 100, preferably at most 80 or at most 50, more preferably at most 25, at most 20, at most 15, at most 10 or at most 5 amino acid differences compared to the mature polypeptide of any one of SEQ ID NOs: 1 or 2.

In another preferred embodiment, the asparaginase is predominantly in a tetramer form.

In another preferred embodiment, the asparaginase is an extracellular asparaginase.

### EXAMPLES

### Materials and Methods

### Asparaginase Activity (ASNU) Assay

The activity of asparaginase may be measured in ASNU. An asparaginase unit (ASNU) is defined as the amount of enzyme needed to generate 1.0 micromole of ammonia in 1 minute at 37° C. and pH 7.0, in 0.1 M MOPS buffer with 9.2 mg/ml asparagine.

Asparaginase hydrolyzes asparagine to aspartic acid and ammonium. The produced ammonium is combined with α-ketoglutarate to form glutamic acid whereby NADH is oxidized to NAD+. The reaction is catalysed by a surplus of glutamate dehydrogenase. The consumption of NADH is measured by photometry at 340 nm. NADH has an absorbance at 340 nm, while NAD+ has no absorbance. A decrease in color is thus measured, and can be correlated to asparaginase activity.

Activity is determined relative to an asparaginase standard of known activity. A commercial product having a declared activity like Acrylaway L may be used as standard.

### Example 1

### Determination of Asparaginase Activity Using Nessler's Reagent

### Principle:

Asparaginase hydrolyses asparagine to aspartic acid and ammonium. Produced ammonium is determined using Nessler's reagent.

### Enzyme Incubation:

| | |
|---|---|
| Temperature | 37° C. |
| pH | 6.0 |
| Buffer | 20 mM citric acid, pH 6 + 0.001% triton x-100 |
| Asparagine substrate sol. | 25 mg/ml in buffer |
| Stop reagent | 1.5M Trichloroacetic acid (TCA) |
| Enzyme concentration | 0 - 4 - 8 ASNU/ml |
| | The enzyme dilutions are made in buffer |
| Incubation time | 10 min |

Enzyme activity was tested in the presence of the following salts added directly to the buffer: CaCl₂, CaSO₄, MgCl₂, MnSO₄, ZnSO₄, and NaCl. Cation concentration in the final enzyme assay solution was: 0-50-100-150-200-250-300 mg cation/L.

### Procedure:

| | |
|---|---|
| Buffer with different salts added | 750 μL |
| Asparagine (25 mg/mL) | 100 μL |
| Sample | 100 μL |
| 1.5M TCA (stop reagent) | 50 μL |
| Total volume | 1.000 μL |

Mix buffer and asparagine and let it equilibrate at 37° C. for 10-15 min. Add enzyme sample and incubate for 10 min at 37° C. Add TCA to stop the reaction.

### Ammonium Determination (Nessler Assay):

| | |
|---|---|
| Temperature | Ambient |
| pH | ~12, controlled by addition of Nessler's reagent |
| Nessler's reagent | Mercury(II)chloride, potassium iodide, potassium |
| | hydroxide |
| Incubation time | 10 min |
| A₄₃₆ | Endpoint measurement at 440 nm |

### Procedure:

| | Microtiter plate |
|---|---|
| MQ Water | 140 μL |
| Sample | 20 μL |
| Nessler's reagent | 40 μL |
| Total volume | 200 μL |

MQ water, sample and Nessler's reagent is added to the microtiter plate. Shake for 10 sec and then leave to incubate for 10 min before shaking again and reading at 440 nm.

Results are compared directly based on reading of the absorbance of the samples.

All samples were run in triplicate.

### Determination of Asparaginase Activity in the Presence of Increasing Amounts of Ions:

The activity of the asparaginase from Aspergillus oryzae (SEQ ID NO: 1), Aspergillus niger (SEQ ID NO: 2) and Pyrococcus furiosus (WO2008151807) was tested in the presence of increasing amounts of ions using the assay described above. The specific ions were added directly to the buffer using the following different salts: CaCl₂, CaSO₄, MgCl₂, MnSO₄, ZnSO₄, NaCl. Enzyme was tested in two dosages, i.e. 4 and 8 ASNU/ml. A control without enzyme was included to test if the ions had any direct effect on the assay. Results are given as measured absorbance based on both mg ion/L added or mmol ion/L to facilitate a direct comparison.

### Results:

Asparaginase from Aspergillus oryzae

### CaCl₂

TABLE 1
Absorbance of samples with increasing Ca⁺⁺-concentration
and enzyme dosages.

| | | |
|---|---|---|
| | | Enzyme conc. |
| Cation conc. | Cation conc. | ASNU/ml |

| mg Ca⁺⁺/L | mmol Ca⁺⁺/L | 0 | 4 | 8 |
|---|---|---|---|---|
| 0 | 0 | 0.058 | 0.314 | 0.731 |
| 0 | 0 | 0.058 | 0.306 | 0.624 |
| 50 | 1.2 | 0.057 | 0.303 | 0.625 |
| 100 | 2.5 | 0.057 | 0.303 | 0.618 |
| 150 | 3.7 | 0.057 | 0.302 | 0.615 |
| 200 | 5.0 | 0.055 | 0.296 | 0.600 |
| 250 | 6.2 | 0.058 | 0.306 | 0.588 |
| 300 | 7.5 | 0.057 | 0.287 | 0.580 |

Activity of A. oryzae asparaginase was not significantly affected in the presence of Ca⁺⁺ ions (from CaCl₂).

### CaSO₄

TABLE 2
Absorbance of samples with increasing Ca⁺⁺-concentration
and enzyme dosages.

| | | |
|---|---|---|
| | | Enzyme conc. |
| Cation conc. | Cation conc. | ASNU/ml |

| mg Ca⁺⁺/L | mmol Ca⁺⁺/L | 0 | 4 | 8 |
|---|---|---|---|---|
| 0 | 0 | 0.056 | 0.298 | 0.600 |
| 0 | 0 | 0.057 | 0.295 | 0.598 |
| 50 | 1.2 | 0.056 | 0.300 | 0.593 |
| 100 | 2.5 | 0.056 | 0.300 | 0.590 |
| 150 | 3.7 | 0.056 | 0.313 | 0.609 |
| 200 | 5.0 | 0.056 | 0.310 | 0.619 |
| 250 | 6.2 | 0.057 | 0.311 | 0.591 |
| 300 | 7.5 | 0.056 | 0.311 | 0.630 |

Activity of A. oryzae asparaginase was not significantly affected by addition of Ca⁺⁺ ions or SOL₁⁻ ions.

### MgCl₂

TABLE 3
Absorbance of samples with increasing Mg⁺⁺-concentration
and enzyme dosages.

| | | |
|---|---|---|
| | | Enzyme conc. |
| Cation conc. | Cation conc. | ASNU/ml |

| mg Mg⁺⁺/L | mmol Mg⁺⁺/L | 0 | 4 | 8 |
|---|---|---|---|---|
| 0 | 0 | 0.057 | 0.294 | 0.574 |
| 0 | 0 | 0.057 | 0.298 | 0.585 |
| 50 | 2.1 | 0.059 | 0.380 | 0.746 |
| 100 | 4.1 | 0.062 | 0.391 | 0.787 |
| 150 | 6.2 | 0.065 | 0.438 | 0.826 |
| 200 | 8.2 | 0.066 | 0.448 | 0.864 |
| 250 | 10.3 | 0.068 | 0.453 | 0.894 |
| 300 | 12.3 | 0.069 | 0.460 | 0.913 |

Activity of A. oryzae asparaginase increased significantly in the presence of Mg⁺⁺ (from MgCl₂).

### MnSO₄

TABLE 4
Absorbance of samples with varying Mn⁺⁺-concentration
and enzyme dosages.

| | | Enzyme conc. | | | Abs(+ enzyme) − | |
|---|---|---|---|---|---|---|
| Cation conc. | Cation conc. | ASNU/ml | | | Abs (no enzyme) | |
| mg Mn⁺⁺/L | mmol Mn⁺⁺/L | 0 | 4 | 8 | 4 | 8 |
| 0 | 0 | 0.06 | 0.32 | 0.66 | 0.26 | 0.60 |
| 0 | 0 | 0.06 | 0.32 | 0.65 | 0.26 | 0.59 |
| 50 | 0.9 | 0.32 | 0.73 | 1.07 | 0.41 | 0.79 |
| 100 | 1.8 | 0.55 | 0.99 | 1.41 | 0.44 | 0.86 |
| 150 | 2.7 | 0.72 | 1.27 | 1.71 | 0.55 | 0.99 |
| 200 | 3.6 | 0.91 | 1.49 | 1.96 | 0.58 | 1.05 |
| 250 | 4.6 | 1.11 | 1.64 | 2.15 | 0.54 | 1.04 |
| 300 | 5.5 | 1.14 | 1.80 | 2.32 | 0.65 | 1.18 |

The presence of Mn⁺⁺ influenced the ammonium detection assay showing an increased response in the samples without enzyme. To see the direct effect of the Mn⁺⁺ the background values were therefore subtracted from the response of the enzymes samples. As shown in the table the activity of the A. oryzae asparaginase increased significantly in the presence of increasing amounts of Mn⁺⁺ (from MnSO₄).

### NaCl

TABLE 5
Absorbance of samples with increasing
Na⁺-concentration and enzyme dosages.

| | | |
|---|---|---|
| | | Enzyme conc. |
| Cation conc. | Cation conc. | ASNU/ml |

| mg Na⁺/L | mmol Na⁺/L | 0 | 4 | 8 |
|---|---|---|---|---|
| 0 | 0 | 0.057 | 0.303 | 0.620 |
| 0 | 0 | 0.057 | 0.301 | 0.598 |
| 50 | 2.2 | 0.056 | 0.303 | 0.594 |
| 100 | 4.3 | 0.056 | 0.302 | 0.608 |
| 150 | 6.5 | 0.056 | 0.310 | 0.616 |
| 200 | 8.7 | 0.056 | 0.312 | 0.619 |
| 250 | 10.9 | 0.057 | 0.310 | 0.609 |
| 300 | 13.0 | 0.056 | 0.307 | 0.617 |

Activity of A. oryzae asparaginase was not significantly affected by Na⁺ (from NaCl).

### ZnSO₄

TABLE 6
Absorbance of samples with increasing Zn⁺⁺-concentration
and enzyme dosages.

| | | |
|---|---|---|
| | | Enzyme conc. |
| Cation conc. | Cation conc. | ASNU/ml |

| mg Zn⁺⁺/L | mmol Zn⁺⁺/L | 0 | 4 | 8 |
|---|---|---|---|---|
| 0 | 0 | 0.056 | 0.247 | 0.454 |
| 0 | 0 | 0.056 | 0.243 | 0.454 |
| 50 | 0.8 | 0.055 | 0.248 | 0.467 |
| 100 | 1.5 | 0.055 | 0.254 | 0.476 |
| 150 | 2.3 | 0.056 | 0.257 | 0.494 |
| 200 | 3.1 | 0.057 | 0.270 | 0.517 |
| 250 | 3.8 | 0.055 | 0.273 | 0.521 |
| 300 | 4.6 | 0.056 | 0.281 | 0.545 |

Activity of A. oryzae asparaginase increased slightly in the presence of Zn⁺⁺ ions (from ZnSO₄).

Asparaginase from Aspergillus niger

### CaCl₂

TABLE 7
Absorbance of samples with increasing Ca⁺⁺-concentration
and enzyme dosages.

| | | |
|---|---|---|
| | | Enzyme conc. |
| Cation conc. | Cation conc. | ASNU/ml |

| mg Ca⁺⁺/L | mmol Ca⁺⁺/L | 0 | 4 | 8 |
|---|---|---|---|---|
| 0 | 0 | 0.056 | 0.184 | 0.318 |
| 0 | 0 | 0.056 | 0.181 | 0.328 |
| 50 | 1.2 | 0.055 | 0.189 | 0.336 |
| 100 | 2.5 | 0.058 | 0.188 | 0.340 |
| 150 | 3.7 | 0.053 | 0.181 | 0.312 |
| 200 | 5.0 | 0.063 | 0.202 | 0.354 |
| 250 | 6.2 | 0.048 | 0.196 | 0.356 |
| 300 | 7.5 | 0.057 | 0.206 | 0.384 |

Activity of A. niger asparaginase showed a slight increase in activity in the presence of Ca⁺⁺ ions (from CaCl₂).

### MgCl₂

TABLE 8
Absorbance of samples with increasing Mg⁺⁺-concentration
and enzyme dosages.

| | | |
|---|---|---|
| | | Enzyme conc. |
| Cation conc. | Cation conc. | ASNU/ml |

| mg Mg⁺⁺/L | mmol Mg⁺⁺/L | 0 | 4 | 8 |
|---|---|---|---|---|
| 0 | 0 | 0.057 | 0.183 | 0.318 |
| 0 | 0 | 0.057 | 0.186 | 0.319 |
| 50 | 2.1 | 0.059 | 0.223 | 0.411 |
| 100 | 4.1 | 0.066 | 0.260 | 0.499 |
| 150 | 6.2 | 0.06 | 0.255 | 0.504 |
| 200 | 8.2 | 0.713 | 0.295 | 0.537 |
| 250 | 10.3 | 0.593 | 0.315 | 0.615 |
| 300 | 12.3 | 0.069 | 0.332 | 0.653 |

Activity of A. niger asparaginase increased significantly in the presence of Mg⁺⁺ (from MgCl₂).

Asparaginase from Pyrococcus furiosus

### CaCl₂

TABLE 9
Absorbance of samples with increasing Ca⁺⁺-concentration
and enzyme dosages.

| | | |
|---|---|---|
| | | Enzyme conc. |
| Cation conc. | Cation conc. | ASNU/ml |

| mg Ca⁺⁺/L | mmol Ca⁺⁺/L | 0 | 4 | 8 |
|---|---|---|---|---|
| 0 | 0 | 0.060 | 0.244 | 0.446 |
| 0 | 0 | 0.060 | 0.241 | 0.445 |
| 50 | 1.2 | 0.057 | 0.231 | 0.427 |
| 100 | 2.5 | 0.065 | 0.238 | 0.445 |
| 150 | 3.7 | 0.069 | 0.233 | 0.408 |
| 200 | 5.0 | 0.061 | 0.224 | 0.394 |
| 250 | 6.2 | 0.054 | 0.203 | 0.363 |
| 300 | 7.5 | 0.061 | 0.209 | 0.372 |

Activity of P. furiosus asparaginase dropped in the presence of increasing amounts of Ca⁺⁺ ions (from CaCl₂).

TABLE 10
Absorbance of samples with increasing Ca⁺⁺-concentration
(up to 500 mg/L) and enzyme dosages.

| | | |
|---|---|---|
| | | Enzyme conc. |
| Cation conc. | Cation conc. | ASNU/ml |

| mg Ca⁺⁺/L | mmol Ca⁺⁺/L | 0 | 4 | 8 |
|---|---|---|---|---|
| 0 | 0 | 0.060 | 0.241 | 0.460 |
| 0 | 0 | 0.061 | 0.249 | 0.471 |
| 83 | 2.1 | 0.060 | 0.240 | 0.442 |
| 167 | 4.2 | 0.065 | 0.226 | 0.451 |
| 250 | 6.2 | 0.064 | 0.215 | 0.402 |
| 333 | 8.3 | 0.065 | 0.205 | 0.365 |
| 416 | 10.4 | 0.054 | 0.184 | 0.352 |
| 500 | 12.5 | 0.062 | 0.186 | 0.337 |

Activity of P. furiosus asparaginase dropped slightly in the presence of increasing amounts of Ca⁺⁺ ions (from CaCl₂).

### MgCl₂

TABLE 11
Absorbance of samples with increasing Mg⁺⁺-concentration
(up to 138 mg/L) and enzyme dosages.

| | | |
|---|---|---|
| | | Enzyme conc. |
| Cation conc. | Cation conc. | ASNU/ml |

| mg Mg⁺⁺/L | mmol Mg⁺⁺/L | 0 | 4 | 8 |
|---|---|---|---|---|
| 0 | 0 | 0.061 | 0.242 | 0.447 |
| 0 | 0 | 0.061 | 0.243 | 0.439 |
| 23 | 0.9 | 0.059 | 0.348 | 0.551 |
| 46 | 1.9 | 0.066 | 0.318 | 0.563 |
| 69 | 2.8 | 0.071 | 0.292 | 0.560 |
| 92 | 3.8 | 0.065 | 0.286 | 0.544 |
| 115 | 4.7 | 0.061 | 0.275 | 0.530 |
| 138 | 5.7 | 0.068 | 0.281 | 0.511 |

Activity of P. furiosus asparaginase increased slightly at Mg⁺⁺ concentrations up to 70 mg Mg⁺⁺/L (from MgCl₂), and dropped again at higher ion concentrations.

TABLE 12
Absorbance of samples with increasing Mg⁺⁺-concentration
(up to 300 mg/L) and enzyme dosages.

| | | |
|---|---|---|
| | | Enzyme conc. |
| Cation conc. | Cation conc. | ASNU/ml |

| mg Mg⁺⁺/L | mmol Mg⁺⁺/L | 0 | 4 | 8 |
|---|---|---|---|---|
| 0 | 0 | 0.060 | 0.243 | 0.440 |
| 0 | 0 | 0.060 | 0.250 | 0.481 |
| 50 | 2.1 | 0.064 | 0.314 | 0.636 |
| 100 | 4.1 | 0.071 | 0.319 | 0.590 |
| 150 | 6.2 | 0.068 | 0.288 | 0.552 |
| 200 | 8.2 | 0.070 | 0.284 | 0.526 |
| 250 | 10.3 | 0.064 | 0.253 | 0.519 |
| 300 | 12.3 | 0.072 | 0.252 | 0.467 |

Activity of P. furiosus asparaginase increased slightly at Mg⁺⁺ concentrations up to 50 mg Mg⁺⁺/L (from MgCl₂), and dropped again at higher ion concentrations.

### CONCLUSION

Mg⁺⁺ and Mn⁺⁺ clearly boosted the activity of the fungal asparaginase from A. oryzae or A. niger, Zn⁺⁺ had some effect, while Ca′ had little or no effect, and Na⁺ no effect. Comparing the effect of CaCl₂ and CaSO₄ on A. oryzae asparaginase showed that it was not the anion used that affected enzyme activity. The bacterial asparaginase from P. furiosus showed an initial boost in activity by Mg⁺⁺ at 50-70 mg/L, but above that activity dropped again. For Ca-ions, the enzyme activity dropped with increasing amounts of Ca⁺⁺. Comparing the activity boosting effects also on molar level of the cations, showed that it was the specific cation and not the molar concentration that was important.

1. A method for producing a heat-treated food product comprising:
(a) contacting of a food material with asparaginase in the presence of magnesium or manganese ions at a concentration of about 0.1-12 millimoles per kg; and
(b) heat-treating the asparaginase treated food material to obtain the heat-treated food product.

2. The method of claim 1, wherein the concentration of magnesium or manganese ions is about 0.1-10 millimoles per kg.

3. The method of claim 1, wherein the concentration of magnesium or manganese ions is about 0.2-12 millimoles per kg.

4. The method of claim 1, wherein the concentration of magnesium or manganese ions is about 0.2-10 millimoles per kg.

5. The method of claim 1, wherein step (a) is performed in the presence of magnesium ions.

6. The method of claim 1, wherein the asparaginase is a fungal asparaginase.

7. The method of claim 1, wherein the asparaginase has at least 50% sequence identity to the mature polypeptide of any one of SEQ ID NOs: 1 or 2.

8. The method of claim 1, wherein the asparaginase is obtained from Aspergillus or is a variant of a parent asparaginase obtained from Aspergillus.

9. The method of claim 1, wherein the asparaginase comprises at most 100 amino acid differences compared to the mature polypeptide of any one of SEQ ID NOs: 1 or 2.

10. The method of claim 1, wherein the heat-treated food product is French fries or sliced potato chips, and wherein step (a) is performed by cuts of potatoes being dipped in, incubated in or sprayed with an asparaginase solution comprising magnesium or manganese ions at a concentration of about 1-12 mM.

11. The method of claim 1, wherein the asparaginase solution comprises magnesium or manganese ions at a concentration of about 2-12 mM.

12. The method of claim 1, wherein the heat-treated food product is a potato-based food product and wherein the food material to be treated with asparaginase is mashed potato, a potato-based dough or a suspension of a dehydrated potato product.

13. The method of claim 1, wherein the heat-treated food product is a cereal-based dough product.

14. The method of claim 1, wherein the heat-treated food product is a breakfast cereal.

15. The method of claim 1, wherein the heat-treated food product is roasted coffee beans, and wherein step (a) is performed by green coffee beans, which have optionally been steamed, being soaked in a solution comprising asparaginase and magnesium or manganese ions at a concentration of about 1-12 mM.
